(19) United States
(12) Patent Application Publication
(10) Pub. No.: US 2010/0221313 A1
(43) Pub. Date: Sep. 2, 2010

(54) TRANSDERMAL RESERVOIR PATCH

(75) Inventors: David J. Smith, San Diego, CA (US); Ludwig Weimann, San Diego, CA (US); Ollen Williams, Jr., III, El Cajon, CA (US); Katherine Mary Evans, San Diego, CA (US); Mark John Badria, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: INNOVATIVE PHARMACEUTICALS, LLC, San Diego, CA (US)

(21) Appl. No.: 12/627,924

(22) Filed: Nov. 30, 2009

Related U.S. Application Data
(60) Provisional application No. 61/118,960, filed on Dec. 1, 2008.

(51) Int. Cl. A61F 13/02; A61K 31/445; B32B 37/02; B32B 38/10; B32B 38/04

(52) U.S. Cl. 424/448; 514/329; 156/60; 156/248; 156/250

(57) ABSTRACT

A transdermal reservoir patch includes a first adhesive layer, a second adhesive layer and an occlusive backing layer configured to inhibit transport of a pharmaceutical agent from the first adhesive layer to the second adhesive layer. Methods of manufacturing the transdermal reservoir patch are disclosed.

### CROSS-REFERENCE TO RELATED APPLICATIONS

This application claims priority to U.S. Provisional Application No. 61/118,960, filed Dec. 1, 2008, which is hereby incorporated by reference in its entirety.

### BACKGROUND OF THE INVENTION

1. Field

Aspects of this invention relate to a patch for the dermal or transdermal administration of a pharmaceutical agent. In addition, disclosed herein are methods related to manufacturing a patch for the dermal or transdermal administration of a pharmaceutical agent.

2. Description of the Related Art

Transdermal devices for the delivery of pharmaceutical agents have been used for maintaining health and treating therapeutically a wide variety of ailments. For example, pain relief medications have been delivered with such devices. The treatment of physical pain concerns health care professionals throughout the world. The treatment of chronic pain is particularly challenging because of the frequent need for repeated administration of pain relief medication. Chronic pain is generally considered to be pain that continues a month or more beyond the usual recovery period for an illness or injury or pain that goes on over months or years as a result of a chronic condition. It may be continuous or come and go. It is estimated that chronic pain disables, to some degree, about 86 million Americans. It is regarded as a source of frustration for the health care professionals who care for the patient, and affects the quality of life and economic security not only of the person with pain, but also his or her family. It is estimated that United States business and industry lose about $90 billion annually to sick time, reduced productivity, and direct medical and other benefit costs due to chronic pain among employees. In some cases, repeated administration of the pain relief medication causes sufferers of chronic pain to develop an undesirable tolerance or addiction, creating further health issues for the patient and additional challenges for the health care professional.

There are a number of methods for administering pain relief medications, including oral and parenteral (administered in a manner other than through the digestive tract). Oral administration is most frequently accomplished by formulating the pain relief medication into tablet or syrup and allowing the patient to swallow it. This method is simple, well accepted and relatively painless, but may be problematic for uncooperative patients. Also, there is often a considerable lapse of time between administration of the pain relief medication and its therapeutic effect because of the time needed for gastrointestinal absorption. This time lag is of particular concern when a patient is suffering from severe or chronic pain. Faster administration may be accomplished by direct injection of the pain relief medication, but most people consider the injection itself to be painful and thus undesirable. A transdermal delivery patch for the delivery of fentanyl has been commercialized (DURAGESIC®, Ortho-McNeil), and is described in U.S. Pat. No. 4,588,580, which is hereby incorporated by reference in its entirety. However, existing transdermal delivery systems are not entirely satisfactory.

### SUMMARY OF THE INVENTION

Disclosed herein are transdermal reservoir patches that include a first adhesive layer and a second adhesive that are configured to inhibit transport of a pharmaceutical agent between the adhesive layers. Also disclosed are methods of making transdermal reservoir patches.

An embodiment described herein relates to a transdermal reservoir patch that can include:

a permeable layer having a skin-facing side, a skin-distal side and an outer perimeter;

an occlusive backing layer having a skin-facing side and a skin-distal side, the skin-facing side of the occlusive backing layer being in contact with the skin-distal side of the permeable layer, wherein a portion of the skin-facing side of the occlusive backing layer extends beyond the outer perimeter of the permeable layer and is configured such that a skin-exposed part of the portion of the skin-facing side of the occlusive backing layer is exposed to the skin of a subject mammal upon securing the patch to the skin;

an enclosed reservoir between the permeable layer and the occlusive backing layer, the reservoir containing a therapeutically effective amount of a pharmaceutical agent;

a first adhesive layer on the skin-facing side of the permeable layer, wherein the first adhesive layer is configured to allow the pharmaceutical agent to pass through the first adhesive layer at a pharmaceutically effective rate when the patch is secured to the skin; and

a protective backing layer having a second adhesive layer on a skin-facing side of the protective backing layer, wherein the protective backing layer contacts the skin-distal side of the occlusive backing layer and the second adhesive layer is configured to secure the patch to the skin;

wherein the skin-exposed part of the portion of the skin-facing side of the occlusive backing layer is configured to inhibit transport of the pharmaceutical agent from the first adhesive layer to the second adhesive layer.

Another embodiment described herein relates to a method of making a transdermal reservoir patch that includes:

providing a controlled membrane laminate (CML), comprising:

a first release liner;

a permeable layer having a skin-facing side, a skin-distal side and an outer perimeter, the skin-facing side being laminated to the first release liner; and

a first adhesive layer between the first release liner and the permeable layer,

wherein the permeable layer has a smaller surface area than the first release liner such that a portion of the first release liner extends beyond the outer perimeter of the permeable layer;

placing a therapeutically effective amount of a pharmaceutical agent on the skin-distal side of the permeable layer;

sealing an occlusive backing layer on the skin-distal side of the permeable layer so as to form an enclosed reservoir containing the pharmaceutical agent; wherein a portion of the occlusive backing layer extends beyond the outer perimeter of the permeable layer;

placing a protective backing layer in contact with a skin-distal side of the occlusive backing layer, wherein the protective backing layer has a second adhesive layer on a skin-facing side thereof; and

adhering a portion of the second adhesive layer to the portion of the first release liner extending beyond the outer perimeter of the permeable layer;

wherein a skin-exposed part of the portion of the occlusive backing layer is configured to inhibit transport of the pharmaceutical agent from the first adhesive layer to the second adhesive layer; and wherein the skin-exposed part of the portion of the occlusive backing layer is exposed to the first release liner.

These and other embodiments are described in greater detail below.

### BRIEF DESCRIPTION OF THE DRAWINGS

FIG. 1 is a cross-sectional schematic view of an embodiment of a transdermal reservoir patch.

FIGS. 2A and 2B are top and perspective schematic views, respectively, of an embodiment of a transdermal reservoir patch.

FIG. 3 is a flowchart describing an embodiment of a method of making a transdermal reservoir patch.

FIG. 4 is a schematic view from the permeable layer side of a controlled membrane laminate (CML) after a cutting and removal step.

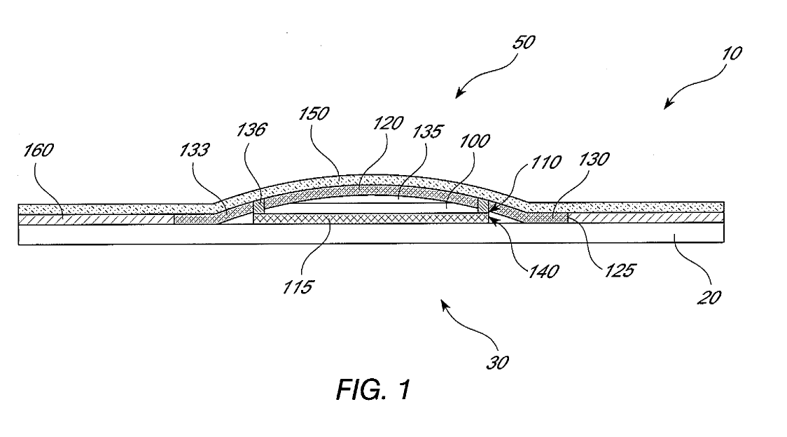

FIGS. 1-4 are not drawn to scale.

### DETAILED DESCRIPTION OF THE PREFERRED EMBODIMENTS

Embodiments disclosed herein relate to the delivery of pharmaceutical agents through a body surface (e.g. skin) involving the use of a transdermal delivery patch that includes a first adhesive region and a second adhesive region, wherein an occlusive backing layer is configured to inhibit transport of a pharmaceutical agent from the first adhesive region to the second adhesive region. Placement of a pharmaceutical agent in a transdermal delivery patch as described herein provides significant benefits, as compared to the use of conventional patches for the delivery of similar compositions. For example, in an embodiment of a transdermal delivery patch which includes two adhesive regions separated by a non-adhesive region, there is a reduced likelihood of the pharmaceutical agents and/or other ingredients leaking out of the transdermal delivery patch and/or wicking out of the transdermal delivery patch during storage and/or usage. In an embodiment of a transdermal delivery patch which includes two adhesive regions separated by an occlusive backing layer, leaks, if any, may be detected more easily. In addition, the extended adhesive area of the second adhesive region provides secure adhesion of the patch to the skin of a patient.

Also, disclosed herein are embodiments of a method of making a transdermal reservoir patch that includes a first adhesive region and a second adhesive region, wherein an occlusive backing layer is configured to inhibit transport of a pharmaceutical agent from the first adhesive region to the second adhesive region.

In describing the present invention, the following terms are to be used as indicated below. As used in this specification and the appended claims, the singular forms “a,” “an” and “the” include plural references unless the content clearly dictates otherwise.

As used herein, the term “transdermal” refers to the use of skin, mucosa, and/or other body surfaces as a portal for the administration of drugs by topical application of the drug thereto for passage into the systemic circulation. Such passage can take place through intact surface (such as skin) without wounds or punctures.

As used herein, the terms “pharmaceutical agent,” “drug,” and “active ingredient” refer to any material that is intended to produce some biological, beneficial, therapeutic, or other intended effect, such as relief of pain, whether or not approved by a government agency for that purpose.

As used herein, the term “therapeutically effective” refers to the amount of drug or the rate of drug administration needed to produce the desired therapeutic result.

### Transdermal Reservoir Patch

FIG. 1 is a cross-sectional schematic view of an embodiment of the transdermal reservoir patch described herein. The patch 10 is configured to be secured to a substrate 20. The substrate 20 may be a first release liner used for storing the patch, or the skin of a subject mammal that is being treated. The patch 10 has a skin-facing side 30 and a skin-distal side 50, where the skin-facing side 30 is configured to face the skin during treatment. The patch 10 includes a permeable layer 100 having an outer perimeter 110 and a first adhesive layer 115 on the skin-facing side 30. An occlusive backing layer 120 contact portions of the skin-distal side 50 of the permeable layer 100 to form an enclosed reservoir 135 containing a therapeutically effective amount of a pharmaceutical agent (for clarity, the agent itself is not shown in FIG. 1, but those skilled in the art will appreciate that it is within the enclosed reservoir 135). In the illustrated embodiment, the occlusive backing layer 120 and the permeable layer 100 are maintained in contact by a seal 136 (e.g., a seal formed by applying heat and/or pressure). FIG. 1 shows the seal 136 extending through both the occlusive backing layer 120 and the permeable layer 100. The illustrated configuration may result from a heat seal, however other configurations of the seal can be used to maintain the enclosed reservoir. For example, the seal may be formed by an adhesive contacting the skin-distal side 50 of the permeable layer 100 and the skin-facing side 30 of the occlusive backing layer 120. The occlusive backing layer 120 is configured so that a portion 130 of the skin-facing side 30 of the occlusive backing layer 120 extends beyond the outer perimeter 110 of the permeable layer 100. The occlusive backing layer 120 also has an outer perimeter 125. In the illustrated embodiment, the occlusive backing layer 120 includes a skin-exposed part 133 of the portion 130 of the skin-facing side 30 of the occlusive backing layer 120. The first adhesive layer 115 has an adhesive perimeter 140 in the illustrated embodiment that is adjacent to the skin-exposed part 133 of the portion 130 of the skin-facing side 30 of the occlusive backing layer 120, such that that the adhesive layer 115 contacts the substrate 20. In an embodiment, the skin-exposed part 133 contacts the skin during treatment.

Contacting the skin-distal side 50 of the occlusive backing layer 120 is a protective backing layer 150 having a second adhesive layer 160 on the skin-facing side 30. The protective backing layer 150 extends beyond the periphery of the occlusive backing layer 120 to secure the patch 10 to the substrate 20. In an embodiment, at least a portion of the second adhesive layer 160 is interposed between the protective backing layer 150 and the occlusive backing layer 120 (not shown). In another embodiment, substantially all of the skin-facing side 30 of the protective backing layer 150 has a second adhesive layer (not shown).

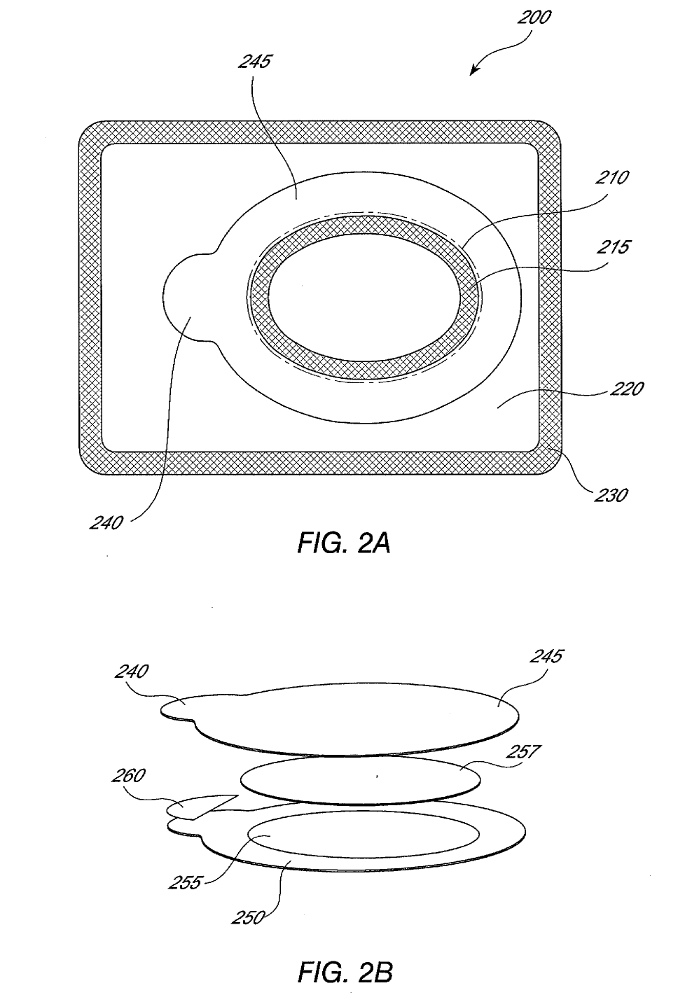

FIG. 2A is a schematic view of another embodiment of a transdermal reservoir patch as viewed from the skin-distal side. The patch 200 depicted in FIG. 2A is generally similar in design to the patch 10. The patch 200 has a portion of the occlusive backing layer 210 extending beyond the outer perimeter of the permeable layer (e.g., the outer perimeter 110 of the permeable layer 100 shown in FIG. 1). A seal 215 may be present to maintain the occlusive backing layer 210 in contact with the permeable layer (e.g., the permeable layer 100 shown in FIG. 1). In one embodiment, the patch 200 is placed within a protective pouch 220. The pouch may optionally have a seal 230. In another embodiment, a peeling means 240, shaped as a “dog ear,” is formed into a portion of the protective backing layer 245 and is configured to ease separation of the patch from the skin and/or first release liner.

FIG. 2B is a perspective schematic view of another embodiment of the transdermal reservoir patch described herein (for clarity, the pouch 220 is not depicted in FIG. 2B). A first release liner 250 contacts both the skin-facing side of the permeable layer 255 and the skin-facing side of the protective backing layer 245. The first release liner 250 may optionally be exposed to a portion of the occlusive backing layer 257. In an embodiment, the first release liner 250 may be in contact with a portion of the occlusive backing layer 257. The first release liner 250 can be separated prior to securing the patch to the skin.

A second release liner 260 may optionally be placed between a portion of the protective backing layer 245 and the first release liner 250 and is configured to ease separation of the patch from the skin and/or first release liner. In an embodiment, at least part of the second release liner 260 is positioned within the peeling means 240.

Although various configurations are disclosed in FIGS. 1 and 2, various other possible designs will be apparent to an individual skilled in the art.

### Permeable Layer

The design of the permeable layer (e.g., as illustrated by the permeable layer 100) may be modified depending on the intended pharmaceutical purpose. Some possible factors include: the rate of delivering the pharmaceutical agent to a subject, the amount of pharmaceutical agent within the patch, where the patch is placed on the subject, etc. In an embodiment, the permeable layer is shaped as an ellipse. In an embodiment, the permeable layer has a surface area in the range of about 1 cm² to about 100 cm², or in the range of about 3 cm² to about 50 cm², e.g., about 5 cm², about 10 cm², about 20 cm², about 30 cm² or about 40 cm². In a preferred embodiment, the permeable layer is an ellipse having a surface area in the range of about 5 cm² to about 40 cm², e.g., about 5 cm², about 10 cm², about 20 cm², about 30 cm² or about 40 cm².

The composition of the permeable layer may include a polymer. Examples of polymers include microporous polyolefin film (e.g., SOLUPOR® from SOLUTECH™), acrylonitrile films, polyethylnapthalene, polyethylene terephthalate (PET), polyimide, polyurethane, polyethylene, polypropylene, ethylene-vinyl acetate (EVA), copolymers thereof and mixtures thereof. In one embodiment, the polymer is EVA. In another embodiment, the polymer is EVA having a vinyl acetate content by weight in the range of about 4% to about 19%. In a preferred embodiment, the polymer is EVA having vinyl acetate content by weight of about 9%.

The permeable layer may also include a heat-sealable material for attaching to other components. As an example, the heat-sealable permeable layer may be an EVA membrane, such as COTRAN™ 9702, available commercially from 3M™.

The thickness of the permeable layer may be varied. For example, the permeable layer thickness may be in the range of about 0.1 mil to about 5 mil, or about 1 mil to about 3 mil. In a preferred embodiment, the thickness is about 2 mil.

### Occlusive Backing Layer

The design of the occlusive backing layer (e.g., as illustrated by the occlusive backing layer 120) is also not particularly limited. In one embodiment, the occlusive backing layer is sized so that a portion of the skin-facing side of the occlusive backing layer extends beyond the outer perimeter of the permeable layer. In another embodiment, the surface area of the occlusive backing layer is larger than that of the permeable layer. In a preferred embodiment, the occlusive backing layer has the same general shape (e.g. ellipse, circle, rectangle, etc.) as the permeable layer but with a slightly larger surface area. The occlusive backing layer may then be optionally configured concentrically with the permeable layer so that the occlusive backing layer extends beyond substantially all of the outer perimeter of the permeable layer within the patch.

The occlusive backing layer may also be configured so as to have a skin-contacting part (e.g., upon application of the skin-exposed part 133) of the portion of the skin-facing side of the occlusive backing layer that is adjacent to the adhesive perimeter (e.g., the perimeter 140) of the first adhesive. In addition, the occlusive backing layer may be configured to have a skin-exposed part of the portion of the skin-facing side of the occlusive backing layer that is adjacent to the adhesive perimeter of the first adhesive. Moreover, the size of these regions may be modified. For example, the outer perimeter of the occlusive backing layer (e.g., as illustrated by the perimeter 125) may extend a distance from the outer perimeter of the permeable layer (e.g., as illustrated by the outer perimeter 110) in the range of about 0.1 mm to about 10 mm, about 0.5 mm to about 5 mm, about 0.5 mm to about 3 mm, about 0.5 mm to about 1.5 mm, or preferably about 1 mm.

In some embodiments, portions of the occlusive backing layer are free of an amount of adhesive that would (if present) be effective to enhance transport of the pharmaceutical agent from the first adhesive layer to the second adhesive layer. In one embodiment, at least the portion of the skin-facing side of the occlusive backing layer is free of an amount of adhesive that would be effective to enhance transport of the pharmaceutical agent from the first adhesive layer to the second adhesive layer. In another embodiment, at least the skin-exposed part (e.g., the skin-exposed part 133) of the portion of the skin-facing side of the occlusive backing layer is free of an amount of adhesive that would be effective to enhance transport of the pharmaceutical agent from the first adhesive layer to the second adhesive layer. In still another embodiment, the skin-contacting part of the portion of the skin-facing side of the occlusive backing layer is free of an amount of adhesive that would be effective to enhance transport of the pharmaceutical agent from the first adhesive layer to the second adhesive layer. In a preferred embodiment, substantially all of the occlusive backing layer is free of an amount of adhesive that would be effective to enhance transport of the pharmaceutical agent from the first adhesive layer to the second adhesive layer.

The occlusive backing layer may suitably include various materials. For example, the occlusive backing layer may include a polymer. Examples of polymers include polyolefin laminates, acrylonitrile films, polyethylnapthalene, polyethylene terephthalate (PET), polyimide, polyurethane, polyethylene, polypropylene, ethylene-vinyl acetate (EVA), copolymers thereof and mixtures thereof. In one embodiment, the occlusive backing layer is a laminate having a skin-facing side and a skin-distal side. The skin-facing side may include a polymer, preferably including EVA and more preferably including EVA having a vinyl acetate content by weight in the range about of about 4% to about 19%, or preferably about 12%. The skin-distal side may also include a polymer, preferably PET. As an example, the occlusive backing layer may be a laminate having polyester and EVA films, such as SCOTCHPAK™ 9733 Backing, available commercially from 3M™.

The occlusive backing layer may also include a heat-sealable material configured for attaching to other components. In a preferred embodiment, the skin-facing side of the occlusive backing layer includes a heat-sealable material configured for attaching to the permeable layer.

The thickness of the occlusive backing layer may also be varied. For example, the permeable layer thickness may be in the range of about 0.5 mil to about 5 mil, or about 1 mil to about 3 mil. In a preferred embodiment, the thickness is about 2 mil.

### Protective Backing Layer

The protective backing layer (e.g., as illustrated by the layer 150) can be formed from any material suitable for making transdermal delivery patches, such as a breathable material (e.g., having MVTR values of at least about 100 g/m²/24 hrs., preferably MVTR values of at least about 500 g/m²/ 24 hrs., or more preferably MVTR values of at least about 700 g/m²/24 hrs.) such as polyurethane film (e.g., CoTran™ 9700 or CoTran™ 9701 available commercially from 3M™) or nonwoven materials (e.g., Softesse™ from DuPont) or occlusive material including, e.g., polyvinyl acetate, polyvinylidene chloride, polyethylene, polypropylene, polyurethane, polyester, ethylene vinyl acetate (EVA), polyethylene terephthalate (PET), polybutylene terephthalate, coated paper products, aluminum sheet and the like, and combinations thereof. In some embodiments, the protective backing layer includes low density polyethylene (LDPE) materials, medium density polyethylene (MDPE) materials or high density polyethylene (HDPE) materials (e.g., SARANEX™ available commercially from Dow Chemical™). In a preferred embodiment, the protective backing layer includes polyurethane.

The backing layer or laminate layer may be a monolithic or a multilaminate layer. In some embodiments, the backing layer and/or laminate layer is a multilaminate layer including nonlinear LDPE layer/linear LDPE layer/nonlinear LDPE layer. The protective backing layer can have a thickness in the range of about 0.5 mil to about 5 mil; more preferably about 1 mil to about 4 mil; or even more preferably about 2 mil or about 3 mil.

### Adhesive Layers

Both the first adhesive layer (e.g., as illustrated by the layer 115) and the second adhesive layer (e.g., as illustrated by the layer 160) may be varied according to different embodiments. Both the first adhesive layer and the second adhesive layer may each independently include a dermatologically acceptable adhesive. Examples of dermatologically acceptable adhesives include, but are not limited to acrylics, natural and synthetic rubbers, ethylene vinyl acetate, poly(alpha-olefins), vinyl ethers, silicones, copolymers thereof and mixtures thereof. In an embodiment, the first adhesive layer includes a silicone adhesive (e.g., BIO-PSA 7-4302 Silicone Adhesive available commercially from Dow Corning®). In another embodiment, the second adhesive layer includes an acrylic adhesive (e.g., DURO-TAK® 87-202A available commercially from Henkel). In a preferred embodiment the first adhesive layer includes a silicone adhesive and the second adhesive layer includes an acrylic adhesive.

In one embodiment, the first adhesive layer is configured to allow the pharmaceutical agent to pass through at a pharmaceutically effective rate when the patch is secured to the skin. In another embodiment, the second adhesive layer is free of a therapeutically effective amount of the pharmaceutical agent.

### Peeling Means

The geometry and placement of the peeling means (e.g., as illustrated by the peeling means 240) can be modified. For example, the peeling means may be positioned on the patch furthest away from the center of the enclosed reservoir (e.g., as illustrated by the enclosed reservoir 135). Also, the peeling means may optionally be shaped similar to a semi-circle or half-ellipse. In one embodiment, the surface area of the peeling means is about the same as an adult human thumbprint or fingerprint.

The placement of the second release liner (e.g., as illustrated by the second release liner 260) is also not particularly limited. In a preferred embodiment, the second release liner is at least partially placed within the peeling means as illustrated in FIG. 2B. Also, the second release liner may include markings (e.g., text or symbols) identifying characteristics of the transdermal patch (e.g., lot number, dosage, etc.) or instructions (e.g., text stating “pull to remove,” etc.)

### Release Liner

The transdermal reservoir patch may optionally include one or more release liners (e.g., as illustrated by the first release liner 250 and the second release liner 260) for storage or handling purposes. Many suitable release liners are known within the art. The release liner can be made of a polymeric material that may be optionally metallized. Examples of suitable polymeric materials include, but are not limited to, polyurethane, polyvinyl acetate, polyvinylidene chloride, polypropylene, polycarbonate, polystyrene, polyethylene, polyethylene terephthalate (PET), polybutylene terephthalate, paper, and combinations thereof. In some embodiments, the release liner is siliconized. In some other embodiments, the release liner is coated with fluoropolymer. In a preferred embodiment, the release liner includes PET coated with fluoropolymer (e.g., SCOTCHPAK™ 9744 from 3M™).

The release liner may have varying thicknesses. For example, the release liner thickness may be in the range of about 1 mil to about 10 mil, or about 2 mil to about 6 mil. In a preferred embodiment, the thickness is about 3 mil or about 4.7 mil.

### Pharmaceutical Agents

Embodiments disclosed herein relate to transdermal reservoir patches that have utility in connection with the delivery of one or more pharmaceutical agents. The pharmaceutical agents may be contained within the enclosed reservoir (e.g., as illustrated by the enclosed reservoir 135) Pharmaceutical agents of embodiments disclosed herein can include agonists and/or additional drugs within the broad class normally delivered through body surfaces and membranes, including skin. In general, this includes therapeutic agents in all of the major areas, including, but not limited to, ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha-two-adrenergic agonists, analgesics, antipyretics and anti-inflammatory agents, androgens, local and general anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretic, antiemetic and prokinetic agents, antiepileptic agents, antiestrogens, antifingal agents, antihypertensive agents, antimicrobial agents, antimigraine agents, antimuscarinic agents, antineoplastic agents, antiparasitic agents, antiparkinson's agents, antiplatelet agents, antiprogestins, antischizophrenia agents, antithyroid agents, antitussives, antiviral agents, atypical antidepressants, azaspirodecanediones, barbituates, benzodiazepines, benzothiadiazides, beta-adrenergic agonists, beta-adrenergic antagonists, selective beta-one-adrenergic antagonists, selective beta-two-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, contraceptive agents, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, hematopoietic agents, histamines, histamine antagonists, hormones, 5-hydroxytryptamine antagonists, drugs for the treatment of hyperlipoproteinemia, hypnotics and sedatives, immunosupressive agents, laxatives, methylxanthines, moncamine oxidase inhibitors, neuromuscular blocking agents, organic nitrates, opiod analgesics and antagonists, pancreatic enzymes, phenothiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins and the like, alone or in combination. Basic drugs such as opioids (e.g., fentanyl and analogs: alfentanil, carfentanil, lofentanil, remifentanil, sufentanil, trefentanil, and the like), galanthamine (or galantamine), and the salts of such basic drugs are well suited to be incorporated in the reservoir. Antagonist drugs for fentanyl, its analogs, and the salts thereof include amiphenazole, naltrexone, methylnaltrexone, naloxone, nalbuphine, nalorphine, nalorphine dinicotinate, nalmefene, nadide, levallorphan, cyclozocine and pharmaceutically acceptable salts thereof.

Some embodiments provide a transdermal reservoir patch that includes pharmaceutically effective amounts of an opioid agonist. The term “opioid agonist” is used herein in the ordinary sense and thus includes opiates, opiate derivatives, opioids, and other substances whose effects are mediated by the same receptor, including mixtures thereof. Non-limiting examples of suitable opioid agonists include: alfenanil; allylprodine; alphaprodine; anileridine; benzitramide; benzylmorphine; beta-endorphin; buprenorphine; butorphanol; carfentanil; clonitazene; codeine; cyclazocine; cyclozine, desomorphine; dextromoramide; dezocine; diamorphine; diampromide; dihydromorphine; dimenoxadol; fentanyl; sufentanil; lofentanil; morphine; normorphine; dihydrocodeine; levorphanol; oxycodone; oxycodone; propoxyphene; meperidine; methadone; normethadone; meptazinol; nicomorphine; pentazocine, remifentanil, heroin, morphine-6-glucuronide; nalbuphine; meptazinol; pethidine; hydromorphone; piritramide; nicomorphine; tilidine; tramadol; opium; met-enkaphalin; delta-enkephalin; dynorphin A; peptide F; Leu-enkephalin; N-alpha-acetylmethadone; dihydromorphine; etorphine; and oxymorphone. The transdermal reservoir patch may include mixtures of any of the opioid agonists. Reference herein to the class of opioid agonists or to a particular opioid agonist will be understood to include reference to pharmaceutically acceptable acids, bases and/or salts thereof and mixtures thereof, unless the context clearly indicates otherwise. Non-limiting examples of particularly preferred opioid agonists include fentanyl, hydromorphone, hydrocodone, ketamine, methadone, oxycodone, oxymorphone, propoxyphene, and sulfentanil.

In a preferred embodiment, the opioid agonist is fentanyl (N-(1-(2-phenylethyl)-4-piperidinyl)-N-phenyl-propanamide) or a pharmaceutically acceptable salt thereof. The amount of fentanyl can be in the range of about 0.1 mg to about 20 mg, preferably in the range of about 0.5 mg to about 15 mg, or more preferably in the range of about 1 mg to about 10 mg, e.g., about 1.25 mg, about 2.5 mg, about 5 mg, about 7.5 mg, or about 10 mg.

In some embodiments, the pharmaceutical agent is intermixed and/or dissolved within a solvent in the enclosed reservoir (e.g., as illustrated by the enclosed reservoir 135). The pharmaceutical agent may be either partially or completely dissolved within the solvent. The possible solvents are not particularly limited so long as they are pharmaceutically acceptable. For example, the solvent may be an organic solvent, an aqueous solvent or mixtures thereof. In one embodiment, the organic solvent is an alkanol. In another embodiment, the alkanol is ethanol. A preferred embodiment includes a ethanol/water solvent mixture in the range of about 10/90 to about 90/10 by weight, preferably in the range of about 30/70 to about 80/20. In an embodiment, the pharmaceutical agent is intermixed and/or dissolved within a gelling agent, such as hydroxyethyl cellulose, hydroxypropyl cellulose or other known gelling agents. As an example, the composition within the reservoir can include 0-47% ethanol by weight, 1-10% gelling agent by weight, 0.1-10% pharmaceutical agent (e.g., fentanyl) by weight, and 10-99% (or alternatively the balance) water by weight. A preferred exemplary range for the composition within the reservoir includes 20-35% ethanol by weight, 1-5% gelling agent by weight, 0.1-2% pharmaceutical agent (e.g., fentanyl) by weight, and the balance being water.

In some embodiments, the transdermal reservoir patch is configured to release the pharmaceutical agent at specified rate for a period of time when secured to a subject mammal (e.g., a human). For example, the transdermal reservoir patch may be configured to release pharmaceutical agent at a rate in the range of about 10 to about 40 mcg/hour per 10 cm² of permeable surface area (e.g., surface area of the permeable layer), e.g., about 25 mcg/hour per 10 cm². In an embodiment, the transdermal reservoir patch can release pharmaceutical agent at the specified rate for a period of time in the range of about 24 hrs. to about 5 days, e.g., about 72 hrs.

In another embodiment, the reservoir is free of an amount of an ingredient that is effective to adversely affect the adhesive properties of the first adhesive layer and the second adhesive layer. An adhesive is adversely affected when the adhesive properties are degraded to a degree that reduces the therapeutic effectiveness of the patch. Glycerol monoleate is an example of such an ingredient.

### Methods of Manufacture

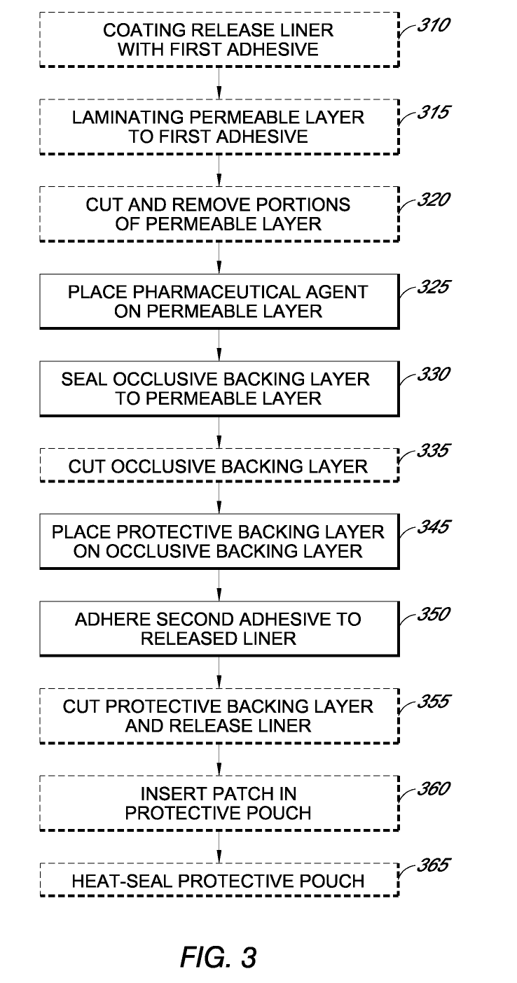

Another embodiment disclosed herein is a method of making a transdermal reservoir patch. FIG. 3 is a flow chart illustrating an embodiment of a method for making a transdermal reservoir patch of the general type illustrated in FIGS. 1 and 2. Optionally, a controlled membrane laminate (CML) is made by coating a first release liner with a first adhesive layer at step 310 followed by laminating a permeable layer forming sheet to the first adhesive layer at step 315. In another embodiment, the first release liner, the permeable layer forming sheet and the first adhesive layer are laminated together having the first adhesive layer in between the first release liner and permeable layer. In still another embodiment, a pre-made CML is provided. The CML may be prepared, for example, using coating equipment from Werner Mathis.

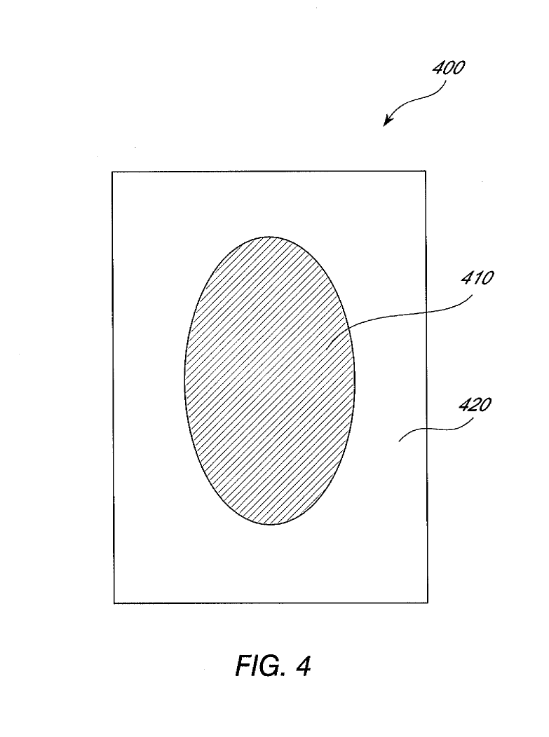

The permeable layer forming sheet may also be cut into a closed shape and portions of the permeable layer forming sheet and first adhesive layer that are peripheral to the closed shape are removed at step 320. In one embodiment, the cutting of the permeable layer forming sheet includes kiss-cutting, e.g., cutting through the permeable layer forming sheet, while cutting a partial depth, if at all, into the release liner. FIG. 4 schematically illustrates a CML 400 as viewed from the permeable layer side after the cutting and removal at step 320. The permeable layer 410 is a closed shape, such as an ellipse, that is smaller than the first release liner 420 such that portions of the first release liner 420 are exposed. Step 320 may be performed, for example, using a semi-automated Rohrer R750 Blister and Patch Development and Production Machine that is adapted for cutting and removing portions of the permeable layer.

Returning to FIG. 3, a pharmaceutical agent is placed on the permeable layer at step 325 and the occlusive backing layer (e.g., as illustrated by the occlusive backing layer 120) is sealed to the permeable layer to form an enclosed reservoir containing the pharmaceutical agent 330. In one embodiment, the sealing of the occlusive backing layer to the permeable layer includes heat-sealing. In another embodiment, the occlusive backing layer can be sealed such that a portion of the occlusive backing layer extends beyond the outer perimeter of the permeable layer. Steps 325 and 330 may be completed using a Rohrer R550 Blister and Patch Development Machine adapted for dispensing the pharmaceutical agent and heat-sealing to form an enclosed reservoir.

In optional step 335, the occlusive backing layer is cut to a desired shape after sealing at step 330. In one embodiment, the cutting of the occlusive backing layer includes kiss-cutting, e.g., cutting through the occlusive packing layer, while cutting a partial depth, if at all, into the release liner. A protective backing layer having a second adhesive layer (e.g., as illustrated by the protective backing layer 150 and the second adhesive layer 160), which optionally has a part of the second adhesive layer adhered to a second release liner (e.g., as illustrated by the second release liner 260), is placed over the occlusive backing layer at step 345 and the second adhesive layer may also be adhered to the first release liner at step 350. The order of steps 345 and 350 is not limited and thus, for example step 350 may occur after step 345, or at about the same time. In one embodiment, the second adhesive layer is free of a therapeutically effective amount of the pharmaceutical agent.

The patch then may be cut into a desired shape at step 355 after the protective backing layer is adhered to the occlusive backing layer, such that the protective backing layer and first release liner are cut at about the same time. If the second release liner is present, the patch may be cut so that the second release liner attached to the protective backing layer is only present within the peeling means of the patch (e.g., as illustrated by the second release liner 260 in FIG. 2). In one embodiment, the patch is placed within a protective pouch at step 360 and the pouch may optionally be heat-sealed at step 365.

The patch may optionally be cured during the manufacturing process (not shown). The curing step may include storing the patch for a period of time (e.g., about 2 weeks) to allow sufficient distribution of the pharmaceutical agent in the enclosed reservoir and the permeable layer.

It will be appreciated that various changes can be made to the manufacturing process described herein without departing from the scope of the present application. For example, some of the process steps may be reordered or occur at about the same time. Also, additional processes can be included, such as sterilizing the patch.

Example 1 below provides an exemplary manufacturing process for the transdermal reservoir patches of embodiments described herein, which is not in any way intended to limit the scope of the claims.

### EXAMPLE 1

### Manufacturing Process for a Transdermal Reservoir Patch

A controlling membrane laminate (CML) is made comprising a first release liner, a silicone adhesive and 9% EVA permeable layer forming sheet. This process is done on a coater equipped with a coating head, drying oven and a lamination station.

The permeable layer forming sheet is kiss cut to a desired shape (e.g., ovals or squares). This process is done on a rotary die cutting machine. The portions of the permeable layer and the first adhesive layer peripheral to the cut shape are removed.

Thixotropic Fentanyl solution is dispensed onto the precut permeable layer (9% EVA) and the solution is confined into the reservoir by means of heat sealing the occlusive backing layer onto the permeable layer around the periphery. This process is done using Hamilton dispensing apparatus and a precision press equipped with the heat seal tooling.

The heat sealed occlusive film is kiss cut into a slightly larger shape than the dimension of the piece of the controlled membrane laminate so that the edges of the occlusive heat sealable film extend about 2 mm away from the edges of the piece of the controlled membrane. This process is done using a precision press with kiss cutting tooling.

The patch is overlayed with a protective backing layer in the form of a 2 mil polyurethane film having a dermatologically acceptable adhesive on one side. The protective backing layer is manufactured with part of the second adhesive layer exposed and part of the second adhesive layer adhered to a second release liner. Both the first release liner and the protective backing layer are then die cut into a desired shape that allows for the protective backing layer and first release liner to be adhered within about a 1 cm region, or any other size suitable by designation of patch use, extending from the edge of the occlusive backing layer, while leaving the second release liner attached to the protective backing layer only on the “dog ear” part of the patch. This process is done using precision press with die cutting tooling.

The extended adhesive area of the overlay provides secure adhesion of the patch to the skin. In addition the overlay is cut with a “dog ear” that allows for easy peel off of the patch from the first release liner prior to placing the patch on the skin. The second release liner from the “dog ear” may be peeled in order to increase the total area of the protective backing layer adhered to the skin.

While the present invention has been described with reference to certain specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A transdermal reservoir patch, comprising:
a permeable layer having a skin-facing side, a skin-distal side and an outer perimeter;
an occlusive backing layer having a skin-facing side and a skin-distal side, the skin-facing side of the occlusive backing layer being in contact with the skin-distal side of the permeable layer, wherein a portion of the skin-facing side of the occlusive backing layer extends beyond the outer perimeter of the permeable layer and is configured such that a skin-exposed part of the portion of the skin-facing side of the occlusive backing layer is exposed to the skin of a subject mammal upon securing the patch to the skin;
an enclosed reservoir between the permeable layer and the occlusive backing layer, the reservoir containing a therapeutically effective amount of a pharmaceutical agent;
a first adhesive layer on the skin-facing side of the permeable layer, wherein the first adhesive layer is configured to allow the pharmaceutical agent to pass through the first adhesive layer at a pharmaceutically effective rate when the patch is secured to the skin; and
a protective backing layer having a second adhesive layer on a skin-facing side of the protective backing layer, wherein the protective backing layer contacts the skin-distal side of the occlusive backing layer and the second adhesive layer is configured to secure the patch to the skin;
wherein the skin-exposed part of the portion of the skin-facing side of the occlusive backing layer is configured to inhibit transport of the pharmaceutical agent from the first adhesive layer to the second adhesive layer.

2. The transdermal reservoir patch of claim 1, wherein an outer perimeter of the occlusive backing layer extends a distance that is in the range of about 0.1 mm to about 10 mm from the outer perimeter of the permeable layer.

3. The transdermal reservoir patch of claim 1, wherein the first adhesive layer and the second adhesive layer each independently comprise a dermatologically acceptable adhesive selected from the group consisting of acrylics, natural and synthetic rubbers, ethylene vinyl acetate, poly(alpha-olefins), vinyl ethers, silicones, copolymers thereof and mixtures thereof.

4. The reservoir patch of claim 1, wherein the permeable layer has a thickness in the range of about 0.5 mil to about 5 mil.

5. The reservoir patch of claim 4, wherein the permeable layer comprises ethylene-vinyl acetate (EVA) having a vinyl acetate content in the range of about 4% to about 19% by weight.

6. The reservoir patch of claim 1, wherein the occlusive backing layer has a thickness in the range of about 0.5 mil to about 5 mils.

7. The reservoir patch of claim 6, wherein the occlusive backing layer is a laminate having a skin-facing layer and a skin-distal layer, wherein the skin-facing layer of the laminate comprises ethylene vinyl acetate (EVA) having a vinyl acetate content in the range of about 4% to about 19% by weight, and the skin-distal layer of the laminate comprises polyethylene terephthalate (PET).

8. The transdermal reservoir patch of claim 1, wherein the pharmaceutical agent comprises an opioid agonist or a pharmaceutically acceptable salt thereof.

9. The transdermal reservoir patch of claim 8, wherein in the opioid agonist is fentanyl or a pharmaceutically acceptable salt thereof.

10. The transdermal reservoir patch of claim 1, further comprising a peeling means.

11. The transdermal reservoir patch of claim 10, further comprising:
a first release liner contacting at least a portion of the first adhesive layer and at least a portion of the second adhesive layer; and
a second release liner disposed between a portion of the protective backing layer and the first release liner,
wherein the second release liner is configured to ease separation of the first release liner from the first adhesive layer and the second adhesive layer.

12. A method of making a transdermal reservoir patch, comprising:
providing a controlled membrane laminate (CML), comprising:
a first release liner;
a permeable layer having a skin-facing side, a skin-distal side and an outer perimeter, the skin-facing side being laminated to the first release liner; and
a first adhesive layer between the first release liner and the permeable layer,
wherein the permeable layer has a smaller surface area than the first release liner such that a portion of the first release liner extends beyond the outer perimeter of the permeable layer;
placing a therapeutically effective amount of a pharmaceutical agent on the skin-distal side of the permeable layer;
sealing an occlusive backing layer on the skin-distal side of the permeable layer so as to form an enclosed reservoir containing the pharmaceutical agent; wherein a portion of the occlusive backing layer extends beyond the outer perimeter of the permeable layer;
placing a protective backing layer in contact with a skin-distal side of the occlusive backing layer, wherein the protective backing layer has a second adhesive layer on a skin-facing side thereof; and
adhering a portion of the second adhesive layer to the portion of the first release liner extending beyond the outer perimeter of the permeable layer;
wherein a skin-exposed part of the portion of the occlusive backing layer is configured to inhibit transport of the pharmaceutical agent from the first adhesive layer to the second adhesive layer; and wherein the skin-exposed part of the portion of the occlusive backing layer is exposed to the first release liner.

13. The method of claim 12, further comprising the step of making the CML by a process comprising:
providing a composite having the first adhesive interposed between the first release liner and a permeable layer forming sheet;
cutting a closed shape into the permeable layer forming sheet, while leaving the first release liner intact; and
removing portions of the permeable layer forming sheet and the first adhesive layer that are peripheral to the closed shape.

14. The method of claim 13, wherein providing a composite comprises:
coating the first release liner with the first adhesive layer; and
laminating a permeable layer forming sheet on top of the first adhesive layer.

15. The method of claim 13, wherein the cutting of the closed shape comprises kiss-cutting the portion of the occlusive backing layer that extends beyond the outer perimeter of the permeable layer.

16. The method of claim 12, wherein a portion of the second adhesive layer is adhered to a second release liner configured to ease separation of the first release liner from the patch.

17. The method of claim 12, further comprising cutting the protective backing layer and the first release liner at about the same time.

18. The method of claim 12, further comprising cutting the protective backing layer, the second release liner layer and the first release liner at about the same time.
